(19) United States
(12) Patent Application Publication
(10) Pub. No.: US 2024/0242776 A1
(43) Pub. Date: Jul. 18, 2024

(54) BIOMARKER FOR PREDICTING COLORECTAL CANCER PROGNOSIS, AND USE THEREOF

(71) Applicant: ARONTIER CO., LTD., Seoul (KR)

(72) Inventors: Insuk SOHN, Seoul (KR); Changha HWANG, Gyeonggi-do (KR); Jooyong SHIM, Daegu (KR)

(21) Appl. No.: 18/560,096

(22) PCT Filed: May 10, 2022
(86) PCT No.: PCT/KR2022/006694

§ 371 Date: Nov. 9, 2023

(30) Foreign Application Priority Data
May 10, 2021 (KR) 10-2021-0060308

(51) Int. Cl. G16B 25/10; G16B 40/20

(52) U.S. Cl. CPC G16B 25/10; G16B 40/20

(57) ABSTRACT

The present invention relates to a biomarker for predicting colorectal cancer prognosis, and a use of the biomarker and, more specifically, to a biomarker composition for predicting the prognosis of colorectal cancer including the ITGB1, FAP, GPX3, SCARA5 and/or KLF4 genes, a composition and kit for predicting the prognosis of colorectal cancer including the biomarker composition, and a method of providing information for predicting the prognosis of colorectal cancer, which includes measuring an expression level of the genes. The ITGB1, FAP, GPX3, SCARA5, and KLF4 genes according to the present invention show a CAF-specific expression pattern, which is characterized by an increase or decrease in expression in cancer-associated fibroblasts (CAFs) compared to normal fibroblasts, so the genes may be beneficial in predicting the prognosis of colorectal cancer.

### TECHNICAL FIELD

The present invention relates to a biomarker for predicting the prognosis of colorectal cancer and a use thereof, and more particularly, to a biomarker composition for predicting the prognosis of colorectal cancer including the ITGB1, FAP, GPX3, SCARA5 and/or KLF4 genes, a composition and kit for predicting the prognosis of colorectal cancer including the biomarker composition, and a method of providing information for predicting the prognosis of colorectal cancer, which includes measuring an expression level of the genes.

### BACKGROUND ART

The cancer microenvironment, also called the tumor microenvironment (TME), is the whole environment relating to cancer cell proliferation and development, is a concept that encompasses cancer cells as well as fibroblasts, blood vessels, lymphatic vessels, immune cells, the extracellular matrix, and adipose cells, which are present in cancer tissue. Existing cancer therapeutics development and research have focused on destroying cancer cells directly. However, research results continuously show that the generation, growth, and infiltration, and even resistance to anticancer agents of cancer cells occur due to interaction with the cancer microenvironment, so attempts targeting the tumor microenvironment are being made to overcome the shortcomings of existing anticancer agents.

Fibroblasts are the most common cells present in animal connective tissue, and play an important role in wound healing by synthesizing the extracellular matrix and collagen. However, fibroblasts present in the cancer microenvironment are activated in association with cancer, and change their characteristics into cancer-associated fibroblasts (CAFs). These CAFs secrete various growth factors such as FGF2, HGF, TGF-beta, SDF-1, VEGF, and IL-6 outside the cells, creating a favorable microenvironment for cancer cells, and since CAFs promote the growth and infiltration of cancer cells, they directly cause the metastasis of cancer cells and act as a major cause of anticancer agent resistance. In addition, CAFs are also associated with the mechanism of CD8+ T cells, and are known to suppress the mechanism of CD8+ T cells for killing cancer cells, and directly kill CD8+ T cells in an antigen-specific and antigen-dependent manner by expressing ligands such as PD-L2 and FASL (Matthew A Lakins et al., Nat Commun. 2018 Mar. 5, 9(1):948).

These CAFs are found in various carcinomas including colorectal cancer, pancreatic cancer, lung cancer, breast cancer, and stomach cancer (Marahaini Musa et al., Future Oncol. 2020 October, 16(29):2329-2344; Tongyan Liu et el., J Hematol Oncol. 2019 Aug. 28, 12(1):86. Eiman A Alhinai et al., Int J Mol Sci. 2019 Oct. 24, 20(21):5295). Carcinomas with an intensive distribution of CAFs do not respond to anticancer agents. Even when the cancer cell death effect that reduces the size of a tumor appears in response to an anticancer agent but the cancer microenvironment based on CAFs is still created, cancer cells can easily grow, and the possibility of recurrence increases. Therefore, even after treatment has ended, continuous monitoring, which includes the detection of CAFs and the prediction of a potential relapse, is carried out, and through this, efforts are being made to treat recurrent cancer at an early stage, thereby aiming to increase the chances of a successful cure (Korean Patent Publication Nos. 10-2016-0021970 and 10-2012-0065959).

However, so far, a very limited number of CAF genes have been discovered, including α-smooth muscle actin (α-SMA) and fibroblast-specific protein 1 (FSP1). Accordingly, there is also a need to discover various CAF gene markers that can be used in cancer prognosis prediction and to develop a method of predicting a cancer prognosis using them.

### DISCLOSURE

### Technical Problem

The present inventors conducted research to discover cancer-associated fibroblast (CAF) gene markers, and to develop a method for predicting the prognosis of colorectal cancer using these markers. As a result, they confirmed that ITGB1, FAP, GPX3, SCARA5, and KLF4 genes show a CAF-specific expression pattern characterized by an increase or decrease in expression in CAFs compared to normal fibroblasts and experimentally proved that these genes can be used to precisely predict the prognosis of colorectal cancer. Thus, the present invention was completed.

### Technical Solution

The descriptions and embodiments disclosed in the present invention can also be applied to other descriptions and embodiments, respectively. That is, all combinations of various elements disclosed in the present invention fall within the scope of the present invention. In addition, it cannot be said that the scope of the present invention is limited by the detailed description below.

In addition, terms not specifically defined in the specification should be understood to have meanings commonly used in the technical field to which the present invention belongs. In addition, unless clearly defined otherwise in the context, singular expressions include plural forms, and plural expressions include singular forms.

One aspect of the present invention provides a biomarker composition for predicting the prognosis of colorectal cancer, which includes one or more genes selected from the genes consisting of ITGB1, FAP, GPX3, SCARA5, KLF4, and a combination thereof.

The term “biomarker” refers to a molecule quantitatively or qualitatively associated with the presence of a biological phenomenon, and the biomarker for predicting the prognosis of colorectal cancer of the present invention may be a gene that serves as a standard for predicting patients with a good or poor prognosis after the onset of colorectal cancer. Specifically, the biomarker may be one or more selected from the genes consisting of ITGB1, FAP, GPX3, SCARA5, KLF4, and a combination thereof.

The term “integrin beta 1 (ITGB1)” is a cell surface receptor protein encoded by the ITGB1 gene, and is known to form an integrin complex serving as a collagen receptor by bonding with integrin alpha 1 and integrin alpha 2, and to be involved in cell attachment and recognition in various processes including embryonic development, hemostasis, tissue repair, immune responses, and the metastatic spread of tumor cells. Specifically, the ITGB1 gene may be a nucleic acid sequence encoding the amino acid sequence of human-derived ITGB1 or include the same, or the nucleic acid sequence of the NCBI Reference Sequence: NM_002211 or NM_033668 disclosed in NCBI or include the same, and the nucleic acid sequence of ITGB1 may be, but not limited to, a sequence having 80%, 85%, 90%, or 95% or more homology with the nucleic acid sequence of the NCBI Reference Sequence: NM 002211 or NM_033668 disclosed in NCBI or include the same, and includes any nucleic acid sequence that can produce an amino acid having the characteristics or functions of ITGB1. In addition, the amino acid sequence of ITGB1 may be the sequence of the NCBI Reference Sequence: NP_002202, NP_391988 or NP_596867 disclosed in NCBI or include the same, the amino acid sequence of ITGB1 may be a sequence having 80%, 85%, 90%, or 95% or more homology with the sequence of each of the NCBI Reference Sequences: NP_002202, NP_391988, and NP_596867 or include the same, but the present invention is not limited thereto. The amino acid sequence of ITGB1 includes any amino acid sequence exhibiting the characteristics or functions of ITGB1.

The term “fibroblast activation protein alpha (FAP-α)” is a transmembrane protein encoded by the FAP gene, and is known to be involved in the growth of fibroblasts or the control of epithelial-mesenchymal interactions during tissue repair. Specifically, the FAP gene may have a nucleic acid sequence encoding the amino acid sequence of human-derived FAP or include the same, the nucleic acid sequence of the NCBI Reference Sequence: NM_001291807 or NM_004460 disclosed in NCBI or include the same, the nucleic acid sequence of FAP may be a sequence having 80%, 85%, 90%, or 95% or more homology with the nucleic acid sequence of the NCBI Reference Sequence: NM_001291807 or NM_004460 disclosed in NCBI or include the same, but the present invention is not limited thereto. The FAP gene has any nucleic acid sequence that can produce an amino acid exhibiting the characteristics or functions of FAP. In addition, the amino acid sequence of FAP may be the sequence of the NCBI Reference Sequence: NP_001278736 or NP_004451 disclosed in NCBI or include the same, or the amino acid sequence of FAP may be a sequence having 80%, 85%, 90%, or 95% or more homology with the sequence of each of the NCBI Reference Sequences: NP_001278736 and NP_004451 or include the same, but the present invention is not limited thereto. The amino acid sequence of FAP includes any amino acid sequence exhibiting the characteristics or functions of FAP.

The term “GPX3” is glutathione peroxidase 3, which is an enzyme encoded by the GPX3 gene, and is known to protect cells from oxidative damage by promoting the glutathione-mediated reduction of organic hydroperoxide and hydrogen peroxide (H₂O₂). Specifically, the GPX3 gene may be a nucleic acid sequence encoded by the amino acid sequence of human-derived GPX3 or include the same, or may be the nucleic acid sequence of the NCBI Reference Sequence: NM_002084 or NM 001329790 disclosed in NCBI or include the same, and the nucleic acid sequence of GPX3 may be a sequence having 80%, 85%, 90%, or 95% or more homology with the nucleic acid sequence of the NCBI Reference Sequence: NM_002084 or NM 001329790 disclosed in NCBI or include the same, but the present invention is not limited thereto. The nucleic acid sequence of GPX3 includes any nucleic acid sequence that can produce an amino acid exhibiting the characteristics or functions of GPX3. In addition, the amino acid sequence of GPX3 may be the sequence of the NCBI Reference Sequence: NP_001316719 or NP_002075 disclosed in NCBI or include the same, or the amino acid sequence of GPX3 may be a sequence having 80%, 85%, 90%, or 95% or more homology with the sequence of each of the NCBI Reference Sequences: NP_001316719 and NP_002075 or include the same, but the present invention is not limited thereto. The amino acid sequence of GPX3 includes any amino acid sequence exhibiting the characteristics or functions of GPX3.

The term “scavenger receptor class A member 5 (SCARA5)” is a ferritin receptor protein encoded by the SCARA5 gene, and is known to mediate iron delivery that is not dependent on transferrin. Specifically, the SCARA5 gene may be a nucleic acid sequence encoded by the amino acid sequence of human-derived SCARA5 or include the same, or may be the nucleic acid sequence of the NCBI Reference Sequence: NM_173833.6 disclosed in NCBI or include the same, and the nucleic acid sequence of SCARA5 may be a sequence having 80%, 85%, 90%, or 95% or more homology with the nucleic acid sequence of the NCBI Reference Sequence: NM_173833.6 disclosed in NCBI or include the same, but the present invention is not limited thereto. The nucleic acid sequence of SCARA5 includes any nucleic acid sequence that can produce an amino acid exhibiting the characteristics or functions of SCARA5. In addition, the amino acid sequence of SCARA5 may be the sequence of the NCBI Reference Sequence: NP_776194.2 disclosed in NCBI or include the same, and the amino acid sequence of SCARA5 may be a sequence having 80%, 85%, 90%, or 95% or more homology with the sequence of the NCBI Reference Sequence: NP_776194.2 or include the same, but the present invention is not limited thereto. The amino acid sequence of SCARA5 includes any amino acid sequence exhibiting the characteristics or functions of SCARA5.

The term “Kruppel like factor 4 (KLF4)” is a transcription factor encoded by the KLF4 gene, and is known to be involved in the control of the proliferation, differentiation, apoptosis, and reprogramming of somatic cells. Specifically, the KLF4 gene may be a nucleic acid sequence encoded by the amino acid sequence of human-derived KLF4 or include the same, or the nucleic acid sequence of the NCBI Reference Sequence: NM_001314052 or NM_004235 disclosed in NCBI or include the same, and the nucleic acid sequence of KLF4 may be a sequence having 80%, 85%, 90%, or 95% or more homology with the nucleic acid sequence of the NCBI Reference Sequence: NM_001314052 or NM_004235 disclosed in NCBI or include the same, but the present invention is not limited thereto. The nucleic acid sequence of KLF4 includes any nucleic acid sequence that can produce an amino acid exhibiting the characteristics or functions of KLF4. In addition, the amino acid sequence of KLF4 may be the sequence of the NCBI Reference Sequence: NP_001300981 or NP_004226 disclosed in NCBI or include the same, and the amino acid sequence of KLF4 may be a sequence having 80%, 85%, 90%, or 95% or more homology with the sequence of each of NCBI Reference Sequences: NP_001300981 and NP_004226 or include the same, but the present invention is not limited thereto. The amino acid sequence of KLF4 includes any amino acid sequence exhibiting the characteristics or functions of KLF4.

In the present invention, the expression of the ITGB1, FAP, GPX3, SCARA5 and/or KLF4 genes may be increased or decreased in CAFs compared to normal fibroblasts.

The term “normal fibroblast” is a fibroblast present outside a cancer lesion, or inside and/or around a cancer lesion, and refers to a fibroblast not exhibiting cancer-associated characteristics. Normal fibroblasts are known to play a role in maintaining tissue homeostasis and secreting or reorganizing various extracellular matrix (ECM) proteins essential for the exhibition of functions (Twana Alkasalias et al., Int J Mol Sci. 2018 May 21, 19(5):1532). Normal fibroblasts may exhibit a function of suppressing the initiation and metastasis of cancer through intracellular contact, paracrine signaling by a soluble factor, or the mechanism of ECM integrity, but may be converted into CAFs according to an integrated process upon tumor progression. In the specification, “normal fibroblast” may be interchangeably used with a normal tissue resident-MSC like fibroblast (tr-MSCF).

The term “cancer-associated fibroblast (CAF)” is a fibroblast present inside and/or around a cancer lesion, and refers to a fibroblast that has changed characteristics such that cancer cells grow. In terms of the characteristics associated with the growth of cancer cells, CAFs can secrete growth factors promoting the growth and infiltration of cancer cells, such as FGF2, HGF, TGF-beta, SDF-1, VEGF, and IL-6, inhibit the mechanism in which CD8+ T cells kill cancer cells, or directly kill CD8+ T cells. In the specification, “cancer-associated fibroblast” may be used interchangeably with “CAF.”

The ITGB1 and/or FAP genes according to the present invention may have increased expression level(s) in CAFs compared to normal fibroblasts, and the GPX3, SCARA5 and/or KLF4 genes may have decreased expression level(s) in CAFs compared to normal fibroblasts. According to an exemplary embodiment of the present invention, the expression level of the gene may be an mRNA expression level of the gene.

The ITGB1, FAP, GPX3, SCARA5 and/or KLF4 genes has/have low expression level(s) in normal fibroblasts, but high expression level(s) in CAFs, or high expression level(s) in normal fibroblasts but low expression level(s) in CAFs. They exhibit distinct expression levels between normal fibroblasts and CAFs. By confirming the CAF-specific expression pattern shown by the genes, it is possible to confirm whether CAFs are present, and further whether the cancer microenvironment is still present, and accordingly, it can be confirmed whether cancer cells are growing again or whether there is possibility of relapse. In other words, the expression level(s) of the ITGB1, FAP, GPX3, SCARA5 and/or KLF4 genes expressed in normal fibroblasts or CAFs can precisely predict a prognosis in a patient diagnosed with cancer, and provide useful information for setting an appropriate treatment direction.

The term “prognosis prediction” refers to the prediction of a patient's condition or the treatment outcome of a disease, and includes a good prognosis (positive prognosis) or bad prognosis (negative prognosis). The “good prognosis (positive prognosis)” includes the improvement or stabilization of a disease, such as the treatment of a disease, tumor regression, the suppression of progression, and an increased survival rate, and the “bad prognosis (negative prognosis)” includes disease progression such as tumor growth, tumor immunosuppression, drug resistance, anticancer agent resistance, cancer metastasis, cancer relapse, and a reduced survival rate. In the specification, “prognosis prediction” may be used interchangeably with “prognosis diagnosis.”

Another aspect of the present invention provides a composition for predicting the prognosis of colorectal cancer, which includes an agent for measuring an mRNA or protein expression level of the gene.

In the composition for predicting the prognosis of colorectal cancer according to the present invention, unless particularly stated otherwise, each term has the same meaning as described in the biomarker composition for predicting the prognosis of colorectal cancer.

Since the composition for predicting the prognosis of colorectal cancer according to the present invention may detect the CAF-specific expression pattern of each gene, it may be used to predict a positive or negative prognosis during cancer treatment or after the completion of cancer treatment.

The term “agent for measuring an mRNA or protein expression level of a gene” refers to an agent that can specifically bind to the mRNA or protein of the ITGB1, FAP, GPX3, SCARA5 and/or KLF4 genes to recognize the genes, or can specifically bind to the mRNA to amplify the genes. As a specific example, the agent for measuring the mRNA or protein expression level of a gene may be a primer, probe, compound, peptide, or protein, which can specifically bind to the mRNA, or an antibody, an aptamer, a probe, a compound, a peptide, or an enzyme, which can specifically bind to the protein, but the present invention is not limited thereto.

The agent may be directly or indirectly labeled to measure an expression level of the mRNA or protein. Specifically, for labeling, ligands, beads, radionuclides, enzymes, substrates, cofactors, inhibitors, fluorescers, chemiluminescent substances, magnetic particles, haptens, and dyes may be used, but the present invention is not limited thereto. As a specific example, as the ligands, biotin, avidin, and streptavidin are included, as the enzymes, luciferase, peroxidase, and ß-galactosidase are included, and as the fluorescers, fluorescein, coumarin, rhodamine, phycoerythrin, and sulforhodamine acid chloride (Texas Red) are included, but the present invention is not limited thereto. Most of the known labels may be used as detectable labels, and a suitable label for the purpose of the present invention may be selected by one of ordinary skill in the art.

The term “primer” refers to a nucleic acid sequence having a free 3′-hydroxyl group, and serves as a starting point for mRNA replication. In the present invention, the primer may include a complementary sequence for the mRNA, but may not need to be completely complementary, and can be used as long as it is sufficiently complementary to hybridize. In addition, the sequence and the length of the primer may be appropriately selected by those of ordinary skill in the art and may be modified to improve the specificity and binding strength to the mRNA. A plurality of primers having different base sequences for the same mRNA may be used.

The term “probe” refers to a nucleic acid fragment that is labeled to a nucleic acid molecule such as RNA or DNA, and allows the expression level of the mRNA to be visually confirmed. In the present invention, the probe may include a complementary sequence for the mRNA, but may need not be completely complementary, and can be used as long as it is sufficiently complementary to hybridize. In addition, the probe may be manufactured in the form of DNA, RNA, or a DNA-RNA hybrid, and specifically, in the form of an oligonucleotide probe, a single chain DNA probe, a double chain DNA probe, or an RNA probe, but the present invention is not limited thereto.

The term “antibody” refers to a protein molecule that serves as an antigen receptor that specifically recognizes a specific antigen, and includes all of a polyclonal antibody, a monoclonal antibody, a whole antibody, and an antibody fragment.

The term “aptamer” refers to a nucleic acid molecule that has a stable tertiary structure and is capable of binding to a target molecule with high affinity and specificity. The aptamer may be RNA, DNA, a modified nucleic acid, or a mixture thereof, and have a linear or cyclic shape.

The composition for predicting the prognosis of colorectal cancer of the present invention may be used in an appropriate analysis method for measuring an mRNA expression level of the gene. As a specific example, an analysis method such as PCR, RT-PCR, qPCR, a ligase chain reaction (LCR), nucleic acid sequence-based amplification (NASBA), electrophoresis, DNA sequencing, RNA sequencing, protein sequencing, next generation sequencing (NGS), whole-genome sequencing (WGS), in situ hybridization, DNA microarrays, Northern blotting, Southern blotting, Western blotting, ELISA, radioimmunoassay, immunodiffusion assay, tissue immunostaining, immunoelectrophoresis, immunoprecipitation assay, complement fixation assay, mass spectrometry, protein microarrays, or flow cytometry may be used. The present invention may include any method that can measure the expression level of the gene without limitation, the present invention is not limited thereto.

In addition, the composition of the present invention may further include one or more other components and solutions, which are suitable for the analysis method. As a specific example, for PCR, buffer solutions, DNA polymerase, RNA polymerase, reverse transcriptase, dNTPs (dATP, dTTP, dGTP, and dCTP), a DNase inhibitor, and a RNase inhibitor may be included. For DNA microarrays, reagents, agents, and enzymes for manufacturing fluorescence-labeled probes may be included, and for ELISA, an antibody specific for the protein, and reagents for detecting the bound antibody (labeled secondary antibodies, chromophores, enzymes and their substrates, other materials capable of conjugating with antibodies, etc.) may be included, but the present invention is not limited thereto.

Still another aspect of the present invention provides a kit for predicting the prognosis of colorectal cancer, which includes an agent for measuring an mRNA or protein expression level of the gene.

In the kit for predicting the prognosis of colorectal cancer according to the present invention, unless particularly stated otherwise, each term has the same meaning as described in the biomarker composition for predicting the prognosis of colorectal cancer, and the composition for predicting the prognosis of colorectal cancer.

Since the kit for predicting the prognosis of colorectal cancer according to the present invention may detect the CAF-specific expression pattern of each gene, it may be used to predict a positive or negative prognosis during cancer treatment or after the completion of cancer treatment.

In addition, the kit of the present invention may predict the prognosis of colorectal cancer by measuring the expression level of the gene using the composition for predicting the prognosis of colorectal cancer. Specifically, the kit may be a PCR kit, a DNA chip kit, or a protein chip kit, but the present invention is not limited thereto.

As a specific example, the kit may be a kit including essential factors for PCR. For example, the PCR kit may include the biomarker, primers specific for the biomarker, test tubes or other suitable containers, reaction buffer solutions (with various pHs and magnesium concentrations), dNTP, ddNTP, enzymes such as Taq-polymerase and reverse transcriptase, a DNase inhibitor, a RNAse inhibitor, and deionized water.

As another example, the kit may be a DNA chip kit including essential factors for the DNA chip. The DNA chip kit may include the biomarker, a substrate to which cDNA or an oligonucleotide corresponding to its fragment is attached, and reagents, agents, and enzymes for manufacturing fluorescence-labeled probes.

As still another example, the kit may be a protein chip kit including essential factors for the protein chip. The protein chip may include the biomarker, a substrate to which the biomarker is attached, and one or more antibodies specifically binding to the biomarker.

Yet another embodiment of the present invention provides a method of providing information for predicting the prognosis of colorectal cancer, which includes the following steps:
(a) measuring an mRNA or protein expression level of the gene in CAFs isolated from an individual; and
(b) comparing the expression level in CAFs, measured in step (a), with that of normal fibroblasts.

In the method of providing information for predicting the prognosis of colorectal cancer according to the present invention, unless particularly stated otherwise, each term has the same meaning as described in the biomarker composition for predicting the prognosis of colorectal cancer, and the composition and kit for predicting the prognosis of colorectal cancer.

The method of providing information for predicting the prognosis of colorectal cancer according to the present invention may detect and compare the CAF-specific expression patterns of the genes, thereby predicting a positive or negative prognosis during cancer treatment or after the completion of cancer treatment.

The step (a) is to obtain a measured value quantified by measuring an mRNA or protein expression level of the gene in CAFs isolated from an individual. According to one exemplary embodiment of the present invention, the expression level of the gene may be the mRNA expression level of the gene.

The step (b) is to compare the expression level in CAFs measured in step (a) with that of normal fibroblasts. Here, the expression level in normal fibroblasts may be previously measured and stored in a database or may be separately collected and measured when measuring the expression level in CAFs.

In addition, the method of providing information for predicting the prognosis of colorectal cancer may include (c) determining whether a positive or negative prognosis is predicted by comparing the measured expression levels of the gene.

Specifically, the step (c) may be to predict a negative prognosis when the expression level(s) of one or more of the ITGB1 and FAP genes in CAFs is/are higher than that measured in normal fibroblasts; or when the expression level(s) of one or more of the GPX3, SCARA5, and KLF4 genes measured in CAFs is/are lower than that measured in normal fibroblasts.

Alternatively, the step (c) may be to predict a positive prognosis when the expression level(s) of one or more of the ITGB1 and FAP genes in CAFs is/are lower than that measured in normal fibroblasts; or when the expression level(s) of one or more of the GPX3, SCARA5, and KLF4 genes measured in CAFs is/are higher than that measured in normal fibroblasts.

The term “individual” may include all of individuals who are likely to develop colorectal cancer or have developed colorectal cancer, and individuals who have completely recovered from colorectal cancer, and a human or any non-human animal without limitation. The non-human animal may be a vertebrate, such as a primate, a dog, a cow, a horse, or a pig, or a rodent such as a mouse, a rat, a hamster, or a guinea pig. In the specification, “individual” may be used interchangeably with “subject” or “patient.”

Yet another aspect of the present invention provides a method of providing information for predicting the prognosis of colorectal cancer, which includes the following steps:
(a) measuring an mRNA or protein expression level of the gene in CAFs isolated from an individual;
(b) obtaining a CAF score by applying the value measured in step (a) to a prognosis prediction model expressed as below:

$$CAF score = ( 0.03054 \; 956 \times [ {G}_{TTGB 1} ] ) + ( 0.05298 \; 038 \times [ {G}_{FAP} ] ) + ( 2.444915 \times {10}^{- 8} \times [ {G}_{GPX 3} ] ) + ( - 0.003421177 \times [ {G}_{SCARA 5} ] ) + ( - 0.0090899 \; 12 \times [ {G}_{KLF 4} ] ) ,$$

wherein [Gₙₐₘₑ] indicates the value obtained in step (a); and
(c) predicting a prognosis in the individual by comparing the CAF score obtained in step (b) with a cut-off value.

In the method of providing information for predicting the prognosis of colorectal cancer according to the present invention, unless particularly stated otherwise, each term has the same meaning as described in the biomarker composition for predicting the prognosis of colorectal cancer, and the composition, kit, and method for predicting the prognosis of colorectal cancer.

The method of providing information for the prognosis of colorectal cancer according to the present invention may predict a positive or negative prognosis during cancer treatment or after the completion of cancer treatment by comparing the detected CAF-specific expression patterns of the respective genes.

The step (a) is to obtain a measured value quantified by measuring an mRNA or protein expression level of the gene in CAFs isolated from an individual. According to one exemplary embodiment of the present invention, the expression level of the gene may be an mRNA expression level of the gene.

The step (b) is to obtain a CAF score by applying the measured value obtained in (a) to a prognosis prediction model expressed as below:

$$CAF score = ( 0.03054 \; 956 \times [ {G}_{TTGB 1} ] ) + ( 0.05298038 \times [ {G}_{FAP} ] ) + ( 0.00000002444915 \times [ {G}_{GPX 3} ] ) + ( - 0.003421177 \times [ {G}_{SCARA 5} ] ) + ( - 0.0090899 \; 12 \times [ {G}_{KLF 4} ] ) ,$$

wherein [Gₙₐₘₑ] indicates the measurement value obtained in (a).

The term “CAF score” is a value that becomes the standard for predicting the prognosis of colorectal cancer, and may be obtained from a calculation formula derived by the linear combination of the expression level of each gene measured in CAFs and the expression levels in CAFs and normal fibroblasts with the regression coefficient obtained by applying the least absolute shrinkage and selection operator (LASSO) algorithm to the Cox proportional hazard model. In the specification, “CAF score” may be used interchangeably with “cancer prognosis prediction score.”

[Gₙₐₘₑ] is a value obtained by measuring the expression level of each gene, [G_{ITGB1}] is a value obtained by measuring an mRNA or protein expression level of the ITGB1 gene, [G_{FAP}] is a value obtained by measuring an mRNA or protein expression level of the FAP gene, [G_{GPX3}] is a value obtained by measuring an mRNA or protein expression level of the GPX3 gene, [G_{SCARA5}] is a value obtained by measuring an mRNA or protein expression level of the SCARA5 gene, and [G_{KLF4}] is a value obtained by measuring an mRNA or protein expression level of the KLF4 gene.

The step (c) is to predict a prognosis in the individual by comparing the CAF score obtained in step (b) with a cut-off value. Specifically, the cut-off value may be 0.97.

In addition, in (c), when the CAF score obtained in step (b) is higher than the cut-off value, a negative prognosis may be predicted; and when the CAF score obtained in step (b) is lower than the cut-off value, a positive prognosis may be predicted.

### Advantageous Effects

As the ITGB1, FAP, GPX3, SCARA5, and KLF4 genes according to the present invention show a CAF-specific expression pattern characterized by an increase or decrease in CAFs compared to normal fibroblasts, the genes can be beneficial in predicting the prognosis of colorectal cancer.

### DESCRIPTION OF DRAWINGS

FIG. 1 is a K-M plot that analyzes the 5-year overall survival (OS) rate for a low-risk group exhibiting a positive prognosis of colorectal cancer or a high-risk group exhibiting a negative prognosis of colorectal cancer.

FIG. 2 is a K-M plot that analyzes the 5-year OS rate for a low-risk group exhibiting a positive prognosis of colorectal cancer, a high-risk group exhibiting a negative prognosis of colorectal cancer, and a group with either an early (stage I and II) or late (stage III and IV) pathological stage.

FIG. 3A is a CAF score distribution chart of a low-risk group exhibiting a positive prognosis of colorectal cancer or a high-risk group with a negative prognosis of colorectal cancer.

FIG. 3B is a graph showing standard log-rank statistics for the CAF score of FIG. 3A.

### MODES OF THE INVENTION

Hereinafter, the present invention will be described in further detail with reference to examples. The examples are provided to more specifically explain the present invention, and the scope of the present invention is not limited by these examples.

### Example 1. Discovery of Biomarker for Predicting Cancer Prognosis

### Example 1-1. Discovery of CAF-Specific Gene

Activated CAFs have a major effect on prognosis such as cancer treatment responsiveness, including tumor immunosuppression, anticancer agent resistance, cancer metastasis, and cancer recurrence. Accordingly, a gene showing a specific expression pattern in the activated CAFs was discovered as a biomarker for predicting cancer prognosis.

Specifically, scRNAseq data (10.1038/s41588-020-0636-z; 10.1038/s41422-020-0355-0) previously disclosed for colorectal cancer was collected and processed to compare mRNA expression levels of a gene expressed in CAFs and normal fibroblasts just before transformation into CAFs. Here, perpetually activated CAFs (paCAFs) were referred to as the CAFs, and normal tissue resident-MSC like fibroblasts (tr-MSCFs) were referred to as the normal fibroblasts just before transformation into CAFs. Afterward, genes showing a statistically significant difference in expression level, including 54 genes whose expression level increases and 83 genes whose expression level decreases in CAFs compared to tr-MSCFs as CAF-specific expression patterns, were found.

### Example 1-2. Selection and Verification of Biomarkers Based on CAF-Specific Genes

From the 54 genes and 83 genes exhibiting CAF-specific expression patterns, confirmed in Example 1, a biomarker gene capable of being effectively used in the prediction of colorectal cancer was selected.

Specifically, a specific gene that can be used as a biomarker was selected by applying the least absolute shrinkage and selection operator (LASSO) algorithm to a Cox proportional hazard model, and all patients were divided into two groups including a low-risk group (low risk) and a high-risk group (high risk) using the leave one out cross validation (LOOCV) based on CAFs. By the dichotomization into the low-risk group (low risk) and the high-risk group (high risk), an optimal cut-off was determined using the maximally selected rank statistic from the ‘maxstat’ R package (Hothorn and Lausen, 2003), and a value corresponding to the most significant relationship with survival among all available cut-off values. The effectiveness of the biomarker was verified by comparing the survival distribution between a low-risk group (low risk) and a high-risk group (high risk) selected in the same manner as described above.

As a result, five genes that can be used as biomarkers for predicting colorectal cancer prognosis were selected, and a gene type, whether an expression level increases or decreases compared to that in trMSCFs, and the regression coefficient are shown in Table 1 below.

TABLE 1

| | Whether expression level | Regression |
|---|---|---|
| Gene | increases or decreases | coefficient |
| ITGB1 | Increases | 0.03054956 |
| FAP | Increases | 0.05298038 |
| GPX3 | Decreases | 0.00000002444915 |
| SCARA5 | Decreases | −0.003421177 |
| KLF4 | Decreases | −0.009089912 |

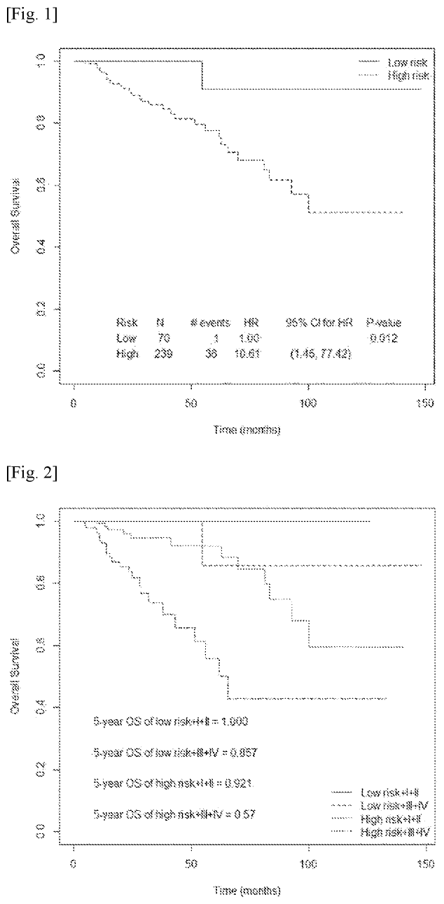

In addition, a total of 309 colorectal cancer samples were classified according to the prognoses in the high-risk group and the low-risk group, or the progression of the cancer at the early and late stages through the comparison of the difference in expression level of the genes. Afterward, Kaplan-Meier (K-M) plots comparing the survival rate between the above groups are shown in FIGS. 1 and 2.

Specifically, FIG. 1 shows whether the prognosis predicted using the five biomarker genes for predicting a prognosis is consistent with a substantial clinical result, and the survival rates of the low-risk group (positive or good prognosis) and the high-risk group (negative or bad prognosis) classified according to the difference in expression level between the genes. The 5-year overall survival rate of the low-risk group is 0.909, which was significantly higher than that of the high-risk group, which is 0.775, and it was confirmed that the hazard ratio (HR) of the high-risk group is 10.61, which is considerably higher than that of the low-risk group (HR: 1.00). In addition, it was confirmed that these hazard ratios are results corresponding to the 5-year overall survival rates.

In addition, FIG. 2 shows whether the prognosis and pathological stages expected using the five biomarker genes for predicting a prognosis correspond to substantial clinical results, and the low-risk group (positive or good prognosis) and the high-risk group (negative or bad prognosis), which are classified according to the difference in expression level between the genes, and the survival rates at stages I to IV. The 5-year overall survival rates per group are shown below, and it was confirmed that this result corresponds to the fact that the survival rates are high in the low-risk group and at an early stage, and low in the high-risk group and at a late stage.
(i) Low hazard, stage I and II groups (low-risk+stage I+stage II): 1.000
(ii) Low hazard, stage III and IV groups (low-risk+stage III+stage IV): 0.857
(iii) High hazard, stage I and II groups (high-risk+stage I+stage II): 0.921
(iv) High hazard, stage III and IV groups (high-risk+stage III+stage IV): 0.557

From the above results, since the selected five biomarker genes (ITGB1, FAP, GPX3, SCARA5, and KLF4) are used to precisely classify and predict colorectal cancer patients according to a prognosis and pathological stage, it can help increase the survival rate of a colorectal cancer patient by providing information for selecting an appropriate treatment method.

### Example 2. Information Provided by Biomarker for Predicting Cancer Prognosis

The types of prognostic information that can be predicted and provided by the biomarker discovered by Example 1 were confirmed.

Specifically, the change in the expression level of a CAF-specific gene was set as a dependent variable (outcome variable), and gene expression, a pathological stage, age, and sex were set as independent variables (explanatory variables), followed by analyzing the influence of each independent variable for the dependent variable. Standard statistical analysis was performed using multivariate Cox regression analysis, and the results are shown in Table 2 below.

TABLE 2

| | Hazard | 95% CI for | |
|---|---|---|---|
| | ratio | hazard ratio | P-value |
| Gene expression level | 14.42 | (1.94, 106.93) | 0.0090 |
| (low vs. high) | | | |
| Stage (I + II vs. III + IV) | 4.27 | (2.02, 9.05) | 0.0002 |
| Age (continuous type) | 1.01 | (0.98, 1.04) | 0.4847 |
| Sex (Male vs. Female) | 1.94 | (0.95, 3.93) | 0.0673 |

As a result, when the expression level of the ITGB1 or FAP gene is higher in CAFs compared to tr-MSCFs, or the expression level of the GPX3, SCARA5, or KLF4 gene is lower in CAFs compared to tr-MSCFs, the hazard rate (relapse rate) increased 14.42-fold compared to the opposite case. In addition, the hazard rate increased 4.27-fold as the cancer progressed from the early stage (stage I and II) to the late stage (stage III and IV), and in terms of age, the hazard rate increased 1.94-fold in men compared to women. However, it was confirmed that there was almost no change in hazard rate, which is 1.01-fold, even when age increases continuously.

From the results, it can be seen that the five biomarker genes (ITGB1, FAP, GPX3, SCARA5, and KLF4) can be used to provide information for predicting the prognosis of colorectal cancer according to gene expression, a pathological stage and sex, except for information on age.

### Example 3. Development and Verification of Cancer Prognosis Prediction Models Using Biomarker for Predicting Cancer Prognosis

Using the biomarkers discovered according to Example 1, as a colorectal cancer prognosis prediction model, a “cancer prognosis prediction score” was developed. Since the biomarkers are genes exhibiting CAF-specific expression patterns, in this Example, “cancer prognosis prediction score” was named “CAF score.”

Specifically, by normalized expression levels of the five biomarker genes (ITGB1, FAP, GPX3, SCARA5, and KLF4) and the linear combination of regression coefficients shown in Table 1, the following CAF score calculation formula was derived:

$$“ CAF score = ( 0.03054 \; 956 \times [ {G}_{TTGB 1} ] ) + ( 0.05298038 \times [ {G}_{FAP} ] ) + ( 2.444915 \times {10}^{- 8} \times [ {G}_{GPX 3} ] ) + ( - 0.003421177 \times [ {G}_{SCARA 5} ] ) + ( - 0.0090899 \; 12 \times [ {G}_{KLF 4} ] ) ”$$

Here, the symbol expressed as “[G_{gene}]” represents the measured expression level of each gene.

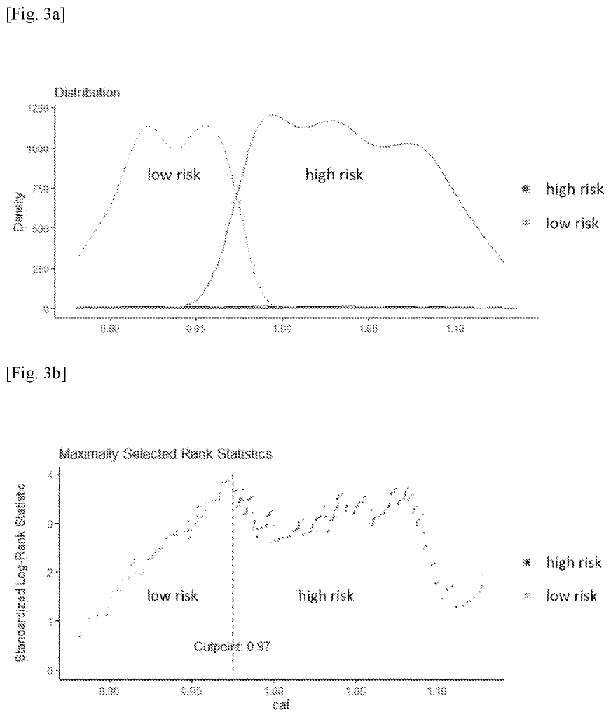

Afterward, the CAF scores were analyzed for colon cancer samples, and their distribution and standard log-rank statistics are shown in FIGS. 3A and 3B.

As a result, as shown in FIGS. 3A and 3B, it was confirmed that the optical cut-off value that distinguishes between the low-risk group and the high-risk group is 0.97. In addition, it was confirmed that the group with a CAF score of less than 0.97 was a low-risk group, and the group with a CAF score of more than 0.97 was a high-risk group, showing, as shown in Example 1-1 and FIG. 1, a result that distinguishes between the low-risk group and the high-risk group.

From the result, the five genes showing CAF-specific expression patterns, and the CAF score using these can be used to effectively perform the prediction of the prognosis of colorectal cancer, such as distinguishing two groups exhibiting different prognoses, so it can be seen that the genes can be useful as biomarkers for predicting colorectal cancer prognosis and colorectal cancer prognosis prediction models.

1. A biomarker composition for predicting the prognosis of colorectal cancer, comprising:
one or more genes selected from a group consisting of ITGB1, FAP, GPX3, SCARA5, KLF4, and a combination thereof.

2. The biomarker composition of claim 1, wherein the expression of the genes is increased or decreased in cancer-associated fibroblasts (CAFs) compared to normal fibroblasts.

3. The biomarker composition of claim 1, wherein the expression of the ITGB1 or FAP gene is increased in CAFs compared to normal fibroblasts.

4. The biomarker composition of claim 1, wherein the expression of the GPX3, SCARA5, or KLF4 gene is decreased in CAFs compared to normal fibroblasts.

5. A composition for predicting the prognosis of colorectal cancer, comprising an agent for measuring an mRNA or protein expression level of the genes of claim 1.

6. The composition of claim 5, wherein the agent for measuring an mRNA expression level of the genes is a primer, a probe, a compound, a peptide, or a protein.

7. The composition of claim 5, wherein the agent for measuring a protein expression level of the genes is an antibody, an aptamer, a probe, a compound, a peptide, or an enzyme.

8. A kit for predicting the prognosis of colorectal cancer, comprising an agent for measuring an mRNA or protein expression level of the genes of claim 1.

9. A method of providing information for predicting the prognosis of colorectal cancer, comprising:
(a) measuring an mRNA or protein expression level of the genes of claim 1 in cancer-associated fibroblasts (CAFs) isolated from an individual; and
(b) comparing the expression level in CAFs measured in step (a) with the expression level in normal fibroblasts.

10. The method of claim 9, further comprising:
(c) predicting a bad prognosis when the expression level(s) of one or more of the ITGB1 and FAP genes measured in CAFs is/are higher than that measured in normal fibroblasts; or when the expression level(s) of one or more of the GPX3, SCARA5, and KLF4 genes measured in CAFs are lower than that measured in normal fibroblasts.

11. The method of claim 9, further comprising:
(c) predicting a good prognosis when the expression level(s) of one or more of the ITGB1 and FAP genes measured in CAFs is/are lower than that measured in normal fibroblasts; or when the expression level(s) of one or more of the GPX3, SCARA5, and KLF4 genes measured in CAFs is/are higher than that measured in normal fibroblasts.

12. A method of providing information for predicting the prognosis of colorectal cancer, comprising:
(a) measuring an mRNA or protein expression level of the genes of claim 1 in CAFs isolated from an individual;
(b) obtaining a CAF score by applying the value measured in step (a) to prognosis prediction models expressed as below:
$$CAF score = ( 0.03054 \; 956 \times [ {G}_{TTGB 1} ] ) + ( 0.05298 \; 038 \times [ {G}_{FAP} ] ) + ( 2.444915 \times {10}^{- 8} \times [ {G}_{GPX 3} ] ) + ( - 0.003421177 \times [ {G}_{SCARA 5} ] ) + ( - 0.0090899 \; 12 \times [ {G}_{KLF 4} ] ) ,$$
wherein [Gₙₐₘₑ] is the measurement value obtained in step (a); and
(c) predicting a prognosis in the individual by comparing the CAF score obtained in step (b) with a cut-off value.

13. The method of claim 12, wherein the cut-off value is 0.97.

14. The method of claim 12, wherein, when the cancer prognosis prediction score obtained in step (b) is higher than the cut-off value, a bad prognosis is predicted.

15. The method of claim 12, wherein, when the cancer prognosis prediction score obtained in step (b) is lower than the cut-off value, a good prognosis is predicted.
